(19) United States
(12) Patent Application Publication
(10) Pub. No.: US 2023/0060942 A1
(43) Pub. Date: Mar. 2, 2023

(54) Health Monitoring System with Precision Eye-Blocking Filter

(71) Applicant: Hypatia Group, Inc., Reston, VA (US)

(72) Inventors: Kelly Marie McVearry, Washington, DC (US); Adam Rankin, Arlington, VA (US); Kenneth Kalunian, San Diego, CA (US)

(21) Appl. No.: 17/873,812

(22) Filed: Jul. 26, 2022

Related U.S. Application Data
(60) Provisional application No. 63/237,388, filed on Aug. 26, 2021.

(51) Int. Cl. G16H 50/20; G06N 20/00

(52) U.S. Cl. CPC G16H 50/20; G06N 20/00; G06V 10/82

(57) ABSTRACT

A method for providing medical services and collecting and monitoring patient data includes taking an image of a patient face and determining position of facial and hair features including eyes and eyebrows. The eyes are masked and the patient is asked to verify that the eyes are masked in order to maintain HIPAA compliance. A health management system with an associated machine learning system can be used to identify facial and hair abnormalities, including rashes and alopecia.

### RELATED APPLICATION

The present disclosure is part of a non-provisional patent application claiming the priority benefit of U.S. patent application Ser. No. 63/237,388, filed on Aug. 26, 2021, which is incorporated by reference in its entirety.

### FIELD OF THE INVENTION

This invention relates generally to the field of remote medical services, including disease or symptom diagnosis and tracking. In some embodiments, data release is subject to various privacy restrictions.

### BACKGROUND

Identification, inspection, tracking, and quantification of disease symptoms and biological status can be an important part of clinical examination and treatment of a patient by healthcare professionals. Systems that can remotely provide for long term symptom tracking can simplify patient management, reduce need for in-person meetings with healthcare professionals, quantify longitudinal quality of life (QOL) measures, and allow for remote participation in clinical trials.

Unfortunately, due to mandated or voluntary patient privacy restrictions, symptom-related data may not always be accessible to clinical trialists and healthcare professionals. For example, under the Health Insurance Portability and Accountability Act of 1996 (HIPAA), sensitive patient health information is not allowed to be disclosed without the patient's consent or knowledge. Specifically, full-face photographs that reveal patient eyes are considered protected health information (PHI) under HIPAA regulations if they can be tied directly to a patient. This can make it difficult to distribute teaching, evaluation, or monitoring images of facial and hair-related symptoms, since such images would typically allow for identification of a patient and their associated medical disease(s). Systems that preserve patient privacy and allow identification, inspection, tracking, and quantification of disease symptoms, as well as being able to store and distribute a photograph of a patient's face in a way that maintains HIPAA-compliance are needed.

### BRIEF DESCRIPTION OF THE DRAWINGS

The specific features, aspects and advantages of the present invention will become better understood with regard to the following description and accompanying drawings where:

FIG. 1 illustrates a system supporting health monitoring conditions that can include remote longitudinal symptom tracking, QOL subtyping, diagnosis, and patient privacy;

FIG. 2 illustrates in more detail an example system supporting measurement of a facial phenotype;

FIG. 3 illustrates in more detail an example system supporting privacy masking of a patient's face by blocking the eyes;

FIG. 4 illustrates one embodiment of a flow chart illustrating user interaction with a system supporting remote diagnosis and patient privacy:

FIG. 5 illustrates another embodiment that allows a desktop, laptop, or mobile application to be used to provide a patient or medical service consumer with a simple, touch centered method and system for providing medical information;

FIG. 6 illustrates another embodiment that provides automated analytics by timescale;

FIG. 7 illustrates another embodiment that provides time dependent symptom frequency information; and

FIG. 8 illustrates another embodiment that provides data collection parameters, timescale, and microreward cost for a custom touch diary mobile application.

### DETAILED DESCRIPTION

In the following disclosure, reference is made to the accompanying drawings, which form a part hereof, and in which is shown by way of illustration specific implementations in which the disclosure may be practiced. It is understood that other implementations may be utilized and structural changes may be made without departing from the scope of the present disclosure. References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

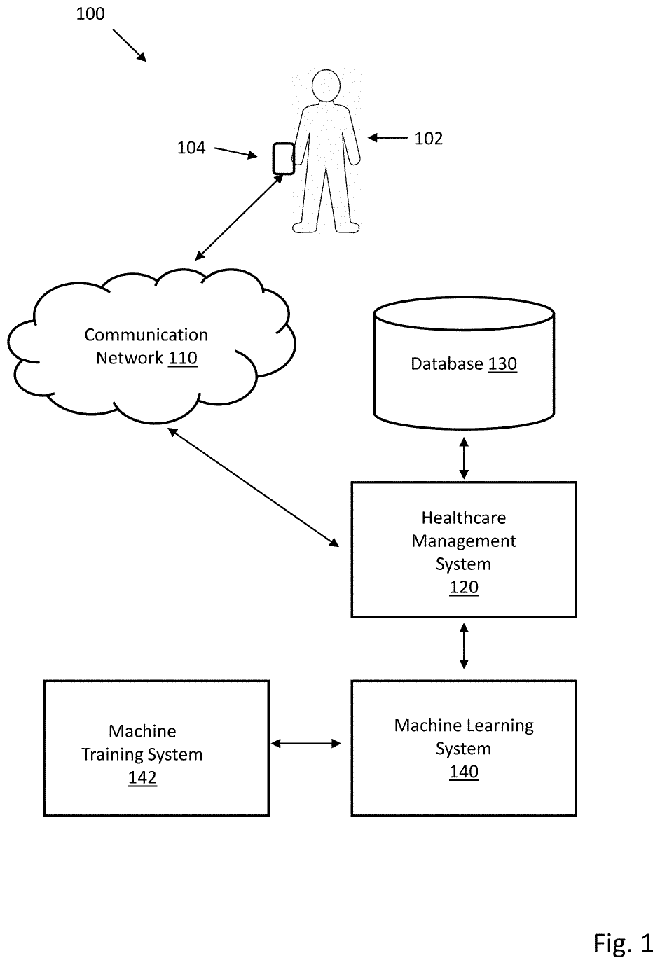

FIG. 1 is a block diagram depicting a system 100 within which an example embodiment may be implemented. A patient 102 with a device 104 can communicate via a communication network 110 with a healthcare management system 120. The healthcare management system 120 can be connected to a database 130 to allow storage and retrieval of medical or other data. The healthcare management system 120 can also be connected to a machine learning system 140 that can act on received data after suitable training with a machine training system 142.

Patient 102 can be one or more individuals needing access to clinical trials or medical services, including longitudinal symptom tracking. In some embodiments patient 102 can be a selected or self-selected member of a group of patients that are members of a treatment group or pool. In other embodiments, patient 102 can provide medical data related to one symptom or condition, and later be selected to be a member of another treatment group or pool.

Device 104 can be any of a wide variety of computing devices, such as a smart watch, a wearable device, smartphone, a desktop computer, a notebook computer, a tablet computer, a server computer, a handheld computer, imagers, digital cameras, and the like. The device 104 can include I/O device(s) and various devices that allow data and/or other information to be input to or retrieved. Example I/O device(s) include cursor control devices, keyboards, keypads, microphones, monitors or other display devices, speakers, printers, or network interface cards, modems, lenses, CCDs or other image capture devices, and the like. Device 104 can also include various interfaces that allow interaction with other systems, devices, or computing environments. For example, device 104 can include any number of different network interfaces, such as interfaces to local area networks (LANs), wide area networks (WANs), wireless networks, and the Internet.

In some embodiments, device 104 includes one or more processors and processing support modules such as data busses, memory device(s), mass storage device(s), and I/O device(s) to communicate with other processing support modules, as well as other coupled devices. Busses can include one or more of several types of bus structures, such as a system bus, graphics bus, PCI bus, IEEE 1394 bus, or a USB bus. Using the processors and processing support modules, device 104 can execute programs or applications to provide for data capture, receipt, analysis, and transmission.

Device 104 can also include multiple sensors able to capture data related to patient 102 or the environment of patient 102. Sensors can include one or more visible, infrared, and/or ultraviolet camera systems. Other sensors can include ultrasonic, infrared, patterned light, or time of flight sensors able to provide three-dimensional data of patient 102. Still other sensors can measure ambient environmental conditions, or patient symptoms such as temperature. The device 104 can be connected to the communication network but can also work independent of connection.

Communication network 110 can include any type of network topology using any communication protocol. Additionally, data communication network 110 may include a combination of two or more communication networks. In some embodiments, data communication network 110 includes a cellular communication network, the Internet, a local area network (LAN), a wide area network (WAN), or any other communication network.

The healthcare management system 120 can be one or more systems that individually or collectively provide medical data collection and analysis services. Data from patient 102 can be analyzed and made available for inspection by healthcare professionals. Hardware supporting operation of the healthcare management system 120 can be similar to that discussed with respect device 104, but can further include use of interconnected computing devices, including one or more of server, desktop, or laptop computers. Interconnect can through different network interfaces, such as interfaces to LANs, WANs, wireless networks, and the Internet.

In some embodiments, the healthcare management system 120 is operable on one or more processors and processing support modules such as data busses, memory device(s), mass storage device(s), and I/O device(s) to communicate with one other processing support modules, as well as other coupled devices. Busses can include one or more of several types of bus structures, such as a system bus, graphics bus, PCI bus, IEEE 1394 bus, or a USB bus. Using the processors and processing support modules, healthcare management system 120 can execute programs or applications to provide for data capture, receipt, analysis, and transmission.

The database 130 is connected to the healthcare management system 120 and can store various information related to symptoms, QOL, medical conditions, and treatments, as well as data related to patients, healthcare professionals, medical data analysts, and the like. Patient data stored in the database 130 can be securely protected through access management, password protection, and encryption. Patient data stored in the database 130 can also be HIPAA-compliant.

The machine learning system 140 is connected to the healthcare management system 130. The machine learning system 140 can use selected data from patient 102 and other sources to provide data analysis of patient symptoms and conditions, with the results being provided to healthcare professionals, data analysts, patients, or others as required through the healthcare management system 120. Various types of machine learning can be used, including supervised, semi-supervised, unsupervised and reinforcement machine learning. Suitable machine learning processing methods include those based on neural networks, naïve bayes, linear regression, logistic regression, random forest, support vector machine (SVM), dimensionality reduction, principal component analysis (PCA), or singular value decomposition (SVD), k-means clustering, or probabilistic clustering methods.

In some embodiments, machine learning system 140 can use one or more neural networks, including fully convolutional, recurrent, generative adversarial, or deep convolutional networks. Convolutional neural networks are particularly useful for image processing applications such as described herein. In some embodiments a convolutional neural network can receive one or more RGB images in RAW, PDF, or JPG format as input. Images can be pre-processed with conventional pixel operations or can preferably be fed with minimal modifications into a trained convolutional neural network. Processing can proceed through one or more convolutional layers, a pooling layer, and a fully connected layer, before output of information related to the image. In operation, one or more convolutional layers can apply a convolution operation to the RGB input, passing the result to the next layer(s). After convolution, local or global pooling layers can combine outputs into a single or small number of nodes in the next layer. Repeated convolutions, or convolution/pooling pairs are possible. In some embodiments, after initial processing is complete, the output can be passed to another neural network for global post-processing with additional neural network-based modifications. All output can be stored in database 130 for later review or use in machine training.

The machine training system 142 is connected to the machine learning system 140. In some embodiments, high quality labeled training data from various sources such as a patient group or pool images, simulated data, or privately or publicly available medical datasets, are prepared for input to a model in the machine training system 142. In one embodiment the machine training system 142 has parameters that can be manipulated to produce desirable outputs for a set of inputs. One such way of manipulating a network's parameters is by “supervised training”. In supervised training, the operator provides source/target pairs to the network and, when combined with an objective function, can modify some or all the parameters of machine training system 142 to provide data sets that guide operation of the machine learning system 140.

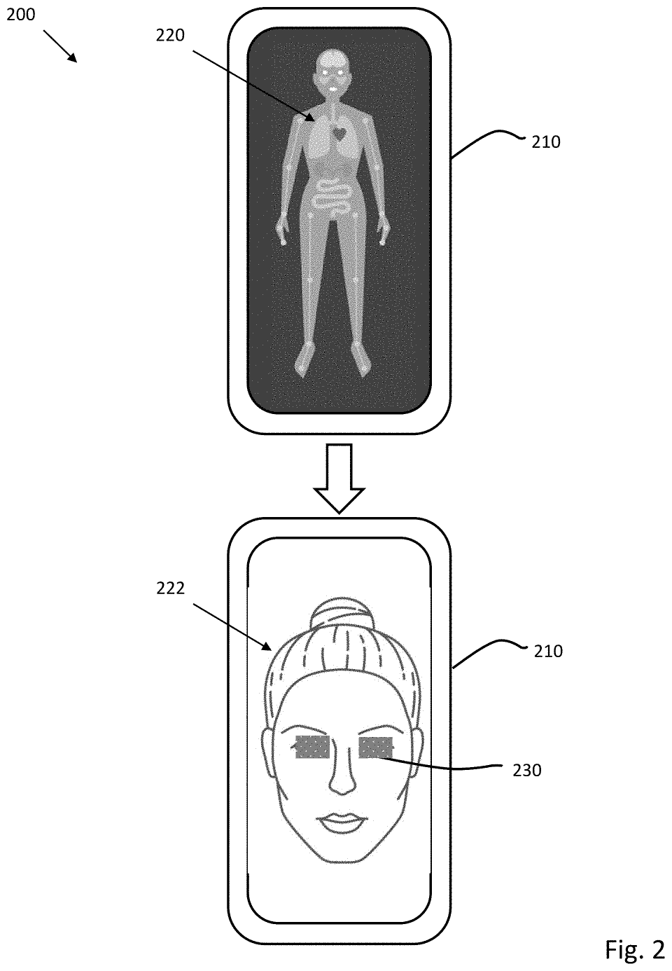

FIG. 2 illustrates in more detail elements of an example system 200 supporting measurement of a facial phenotype. In this embodiment the facial phenotype for lupus, diversity and inclusion phenotyping can be measured. As illustrated, a smartphone 210 provides an imaged body 220 that can be used for single-tap symptom reporting. Such reporting can involve touch based reporting of inflammation and pain across internal and external organs of interest. Rashes or other skin and hair abnormalities can be detected using this single-tap symptom reporting, including butterfly rash and eyebrow or other alopecia often associated with lupus. If a butterfly rash in a patient's face is detected, the patient can be prompted to take a selfie or closeup face image 222.

To be HIPAA-compliant, this selfie or closeup face image can initiate localized masking 230 around each eye of a patient. To preserve medical information, only the eyes are masked, with eyebrows and nasal bridge still being unmasked and visible. Masking can be initiated locally, or after communication with a health management system such as discussed with respect to FIG. 1. In some embodiments all or some of the patient interactions with the smartphone 210 can use single tap user interface of lupus phenotype or other autoimmune symptoms related to disease progression or quality of life.

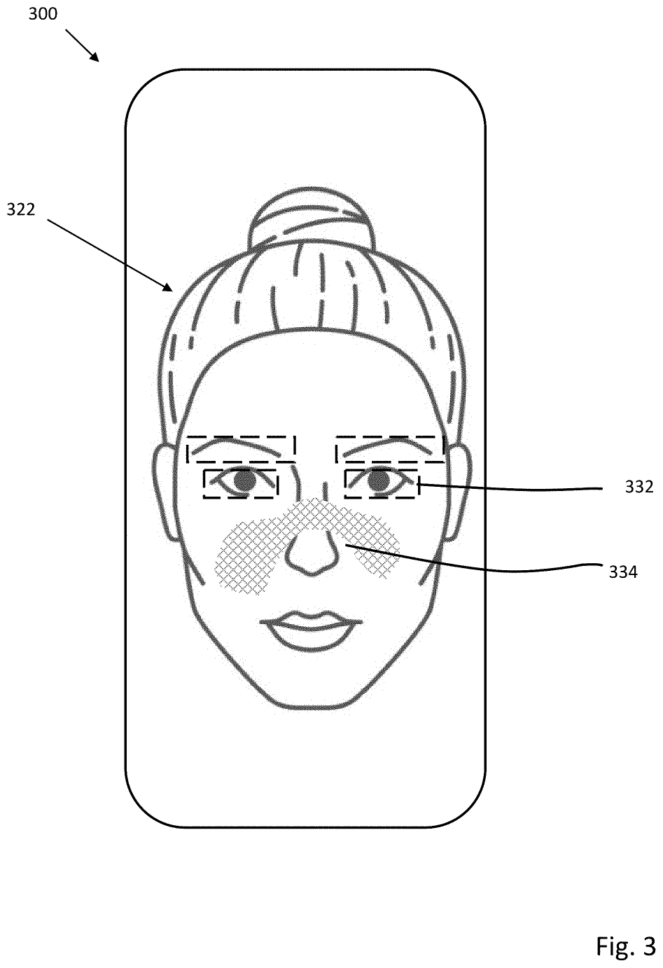

FIG. 3 illustrates in more detail aspects of a health management system image data 300 supporting symptom tracking of a patient's face and machine learning mediated data analysis. In one embodiment, a smartphone (not shown) is used to take a selfie or closeup face image 322 of a patient. In some embodiments, the image 322 can be locally masked using machine learning algorithms executed by the smartphone application, while in other embodiments eye masking is completed by machine learning algorithms remotely executed by the health management system. Next, this masked or unmasked image 322 is sent to the health management system. To improve privacy and maintain HIPAA compliancy, any selfie or closeup face image with eyes showing is encrypted for transmission and not stored by the health management system server, database, or any cloud server. In some embodiments, to increase security, the masked selfie or closeup face image 322 can be marked with a user's public key and added as part of their non-fungible token (NFT) portfolio. In addition, the patient can be provided with the choice of opting out of taking the image 322 entirely or allowing the masked or unmasked image 322 to be used for purposes not related to clinical trials and/or patient monitoring and treatment.

Features such as eyes or eyebrows can be identified and marked with auto-adjusting bounding boxes 332 for later data processing or eye masking, if needed. Rashes or other skin and hair abnormalities 334 can be detected, including butterfly rash (as illustrated) and eyebrow or other alopecia often associated with lupus. Skin tone on a spectrum can also be detected for diversity and inclusion phenotyping. Detection can be using trained machine learning systems, including convolutional neural networks. In some embodiments, when enough data are collected, daily symptom reports and condition tracking can be used to make patient outcome predictions, classify symptoms, warn the patient or other if their conditions may be getting worse. In some embodiments, organizations conducting medical trials and pharmaceutical sponsors can be provided with near real time evidence demonstrating drug efficacy during an exposure period. At scale, with sufficient training data, the disclosed inflammation tracker and condition tracker can function as a companion diagnostic system to improve patient outcome.

Eyebrow bounding boxes, rashes or other skin and hair abnormalities, and skin tone can be stored within the database while maintaining HIPAA-compliance. The eye bounding boxes are blocked from the database for the image to be HIPAA-compliant, but data collected from the eyes that cannot be linked to a single patient can be stored within a curated eye damage library. All data can remain encrypted while maintained in a database or when communicated or otherwise transferred.

For eyebrow alopecia, “nearest neighbor pixel” method can be used, using pixel contrast as machine learning kernel. No pixel contrast will denote no eyebrows and changing pixel contrast will denote loss or gain of eyebrow hair over time. The pixel coordinates are mapping tools for timescale data visualization. Data relating to “drug on” and “drug off” analytics and alopecia impacts can also be collected throughout trials.

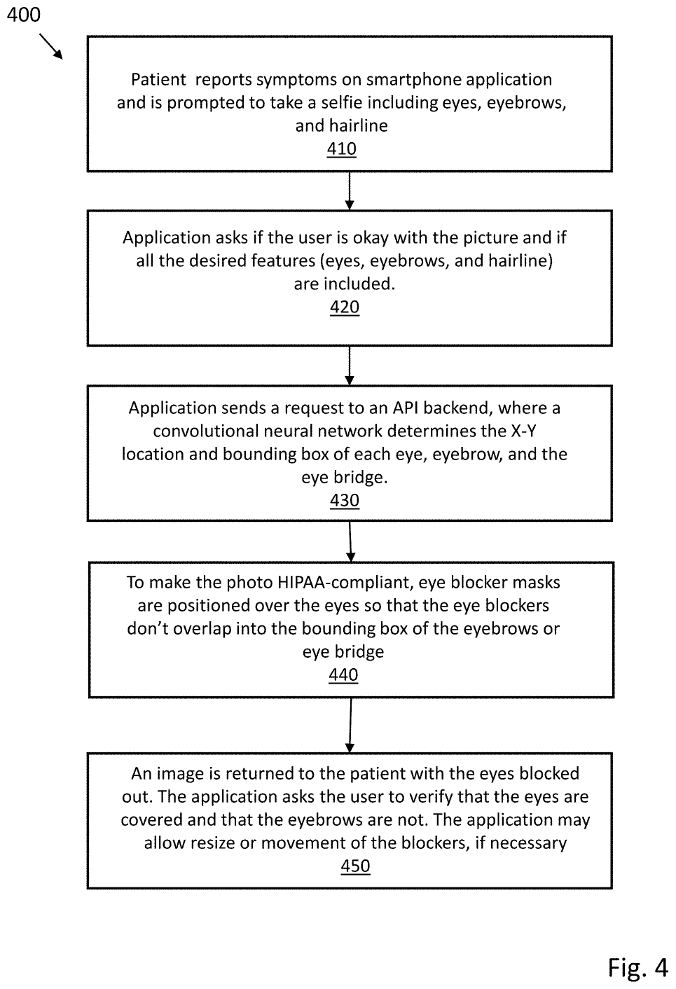

FIG. 4 illustrates one embodiment of a flow chart 400 illustrating user interaction with a system supporting remote symptom tracking, diagnosis, drug implications, and patient privacy for real-time data visualization for quantifying information under HIPAA regulations. In one embodiment, a patient reports symptoms on smartphone application or other interactive unit and is prompted to take a selfie including eyes, eyebrows, and hairline (step 410). The smartphone application asks if the patient is okay with the picture and if all the desired features (eyes, eyebrows, and hairline) are included (step 420). The application sends a request to an API backend of the health management system, where a convolutional neural network in an attached machine learning system determines the pixel-based X-Y location and bounding box of each eye, eyebrow, and the eye bridge (step 430). To make the photo HIPAA compliant, eye blocker masks are positioned over the eyes so that the eye blockers don't overlap into the bounding box of the eyebrows or eye bridge (step 440). An image is returned to the patient with the eyes blocked out. The application asks the user to verify that the eyes are covered and that the eyebrows are not. The application may allow resize or movement of the blockers, if necessary (step 450).

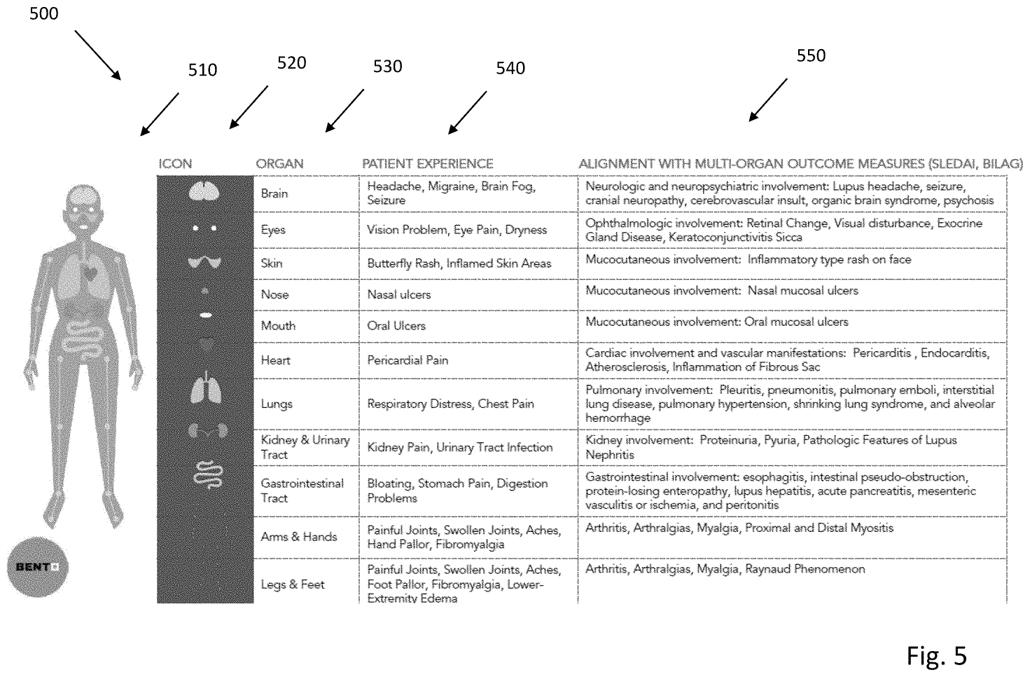

FIG. 5 illustrates another embodiment that allows a desktop, laptop, or mobile application to be used to provide a patient or medical service consumer with a simple, touch centered method and system for providing medical information. This application can provide a touch diary that maps patient symptoms to various clinical multi-organ outcome measures of disease activity (e.g SLEDAI, BILAG, or Wolfe Index). This is illustrated with respect to FIG. 5, which illustrates a touch centric user interface system 500 that includes one or more body illustrations 510, various explanatory image icons 520, and explanatory text 530. Additionally, textual information relating to patient experience 540 and information 550 relating to various clinical multi-organ outcome measures of disease activity (e.g SLEDAI, BILAG, or Wolfe Index) is provided.

In some embodiments, body illustrations 510 can be provided from various viewpoints (including but not limited to front, back or side) or alternatively can be 3D rotating or rotatable illustrations. In some embodiments, a body illustration can be resized larger or smaller, or zoomed in focus on particular selected body areas (e.g head, face, torso, arms, hands, or legs, feet). In some embodiments, important bodily features can be colored or otherwise highlighted. In other embodiments, textual, voice, or graphical cues can be used to encourage touch input to body illustrations 510, as well as providing support for alternative textual or voice input by a patient.

In some embodiments, explanatory image icons 520, and explanatory text 530 can include reference to various organs or body parts. For example, icons can be provided for brain, eyes, skin, nose, mouth, heart, lungs, kidney and urinary tract, gastrointestinal tract, arms, hands, legs, or feet. Other organs or bodily parts can also be included as necessary. In some embodiments, specific body areas can be identified by a patient, user, or medical service provider by touch or verbal explanation. This could include, for example, specific identification of location of major lesions, sores or inflamed skin.

Textual information relating to patient experience 540 can include, but is not limited to headache, migraine, brain fog, seizure, vision problem, eye pain, dryness, butterfly rash, inflamed skin areas, nasal ulcers, oral ulcers, pericardial pain, respiratory distress, chest pain, kidney pain, urinary tract infection, bloating, stomach pain, digestion problems, painful joints, swollen joints, aches, hand pallor, hand related fibromyalgia, foot pallor, foot related fibromyalgia, or lower-extremity edema.

Information 550 relating to various clinical multi-organ outcome measures of disease activity (e.g SLEDAI, BILAG, or Wolfe Index) can include noting neurologic and neuropsychiatric involvement such as lupus headache, seizure, cranial neuropathy, cerebrovascular insult, organic brain syndrome, psychosis, ophthalmologic involvement such as retinal change, visual disturbance, exocrine gland disease, keratoconjunctivitis sicca, mucocutaneous involvement such as inflammatory type rash on face, mucocutaneous involvement such as nasal mucosal ulcers, mucocutaneous involvement such as oral mucosal ulcers, cardiac involvement, vascular manifestations such as pericarditis, endocarditis, atherosclerosis, inflammation of fibrous sac, pulmonary involvement such as pleuritis, pneumonitis, pulmonary emboli, interstitial lung disease, pulmonary hypertension, shrinking lung syndrome, and alveolar hemorrhage, kidney involvement such as proteinuria, pyuria, pathologic features of lupus nephritis, gastrointestinal involvement such as esophagitis, intestinal pseudo-obstruction, protein-losing enteropathy, lupus hepatitis, acute pancreatitis, mesenteric vasculitis or ischemia, or peritonitis, and arthritis, arthralgias, myalgia, proximal and distal myositis, arthritis, arthralgias, myalgia, or Raynaud phenomenon. In some embodiments, multi-organ outcome measures can be provided by a medical service provider, alone or in combination with machine learning diagnostic systems.

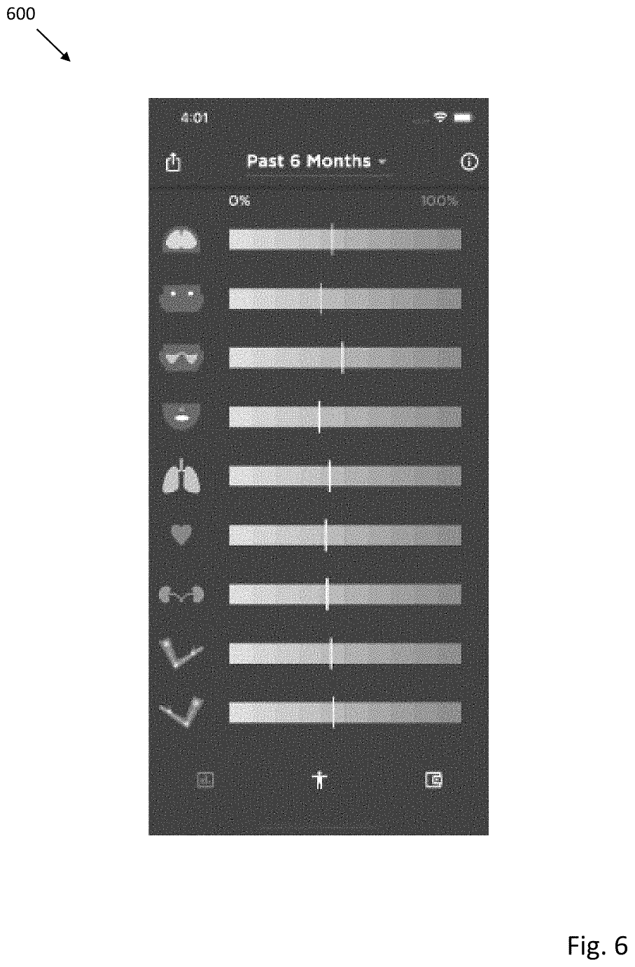

FIG. 6 illustrates another embodiment of a touch diary system 600 that provides a graphical display of user identified symptom frequency tracking for various organs. In some embodiments, a touch button can be used to generate a PDF or other suitably formatted document for archival or other purposes. In some embodiments, user tracked symptom data can be graphically illustrated using a grey scale (e.g. light to dark, with light grey indicating few or no symptoms and darker grey colors respectively illustrating moderate to higher frequency symptom presentation) or color scale (green is few or no symptoms in a selected time period, yellow is some symptoms, and red is high frequency of symptom presentation). In some embodiments, this information can be presented in addition or alone with reference to textual, voice, or other data presentation method.

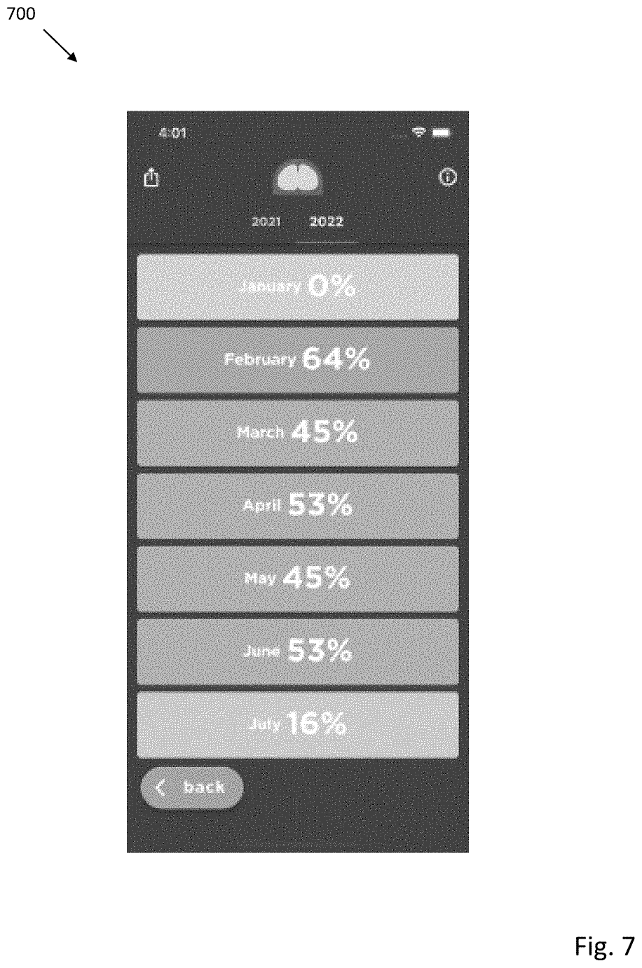

FIG. 7 illustrates another embodiment of a touch diary system 700 that provides a graphical display of user identified symptom frequency tracking for a particular organ. In the illustrated embodiment a screen shows frequency of symptoms associated with this organ over a selected time period. In some embodiments, user tracked symptom data can be graphically illustrated using a grey scale (e.g. light to dark, with light grey indicating few or no symptoms and darker grey colors respectively illustrating moderate to higher frequency symptom presentation) or color scale (green is few or no symptoms in a selected time period, yellow is some symptoms, and red is high frequency of symptom presentation). In some embodiments, this information can be presented in addition or alone with reference to textual, voice, or other data presentation method.

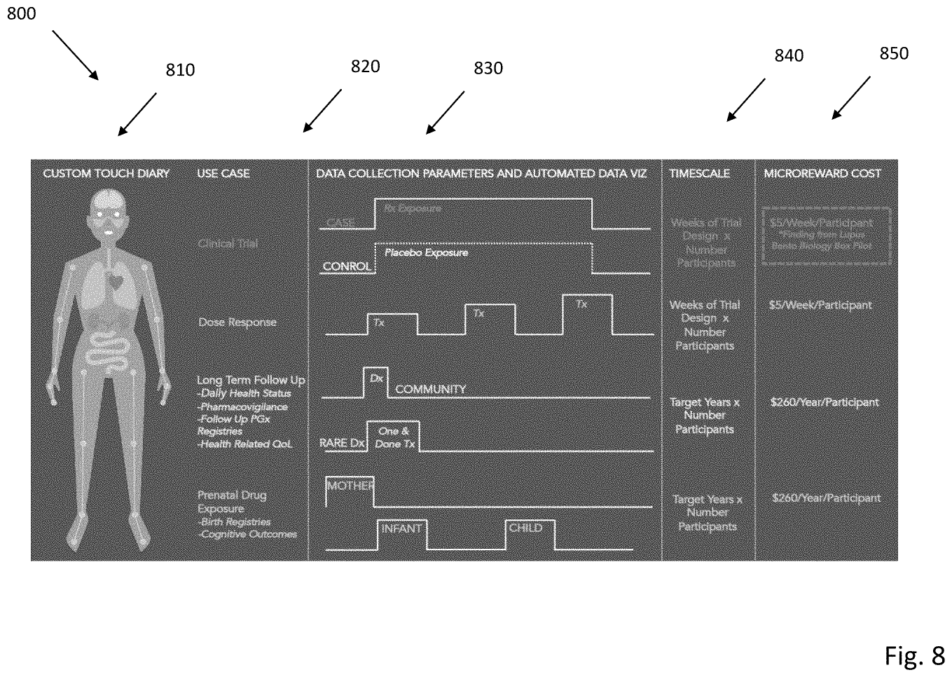

FIG. 8 illustrates another embodiment that provides data collection parameters and automated data visualization, timescale, and microreward cost for a custom touch diary mobile application 800. This application can provide a touch diary that provides information related to various use cases. As seen with respect to FIG. 8, which illustrates a touch centric user interface system 800 can include one or more body illustrations 810, various use cases 820, and data collection and automated data visualization text 830. Additionally, textual information relating to timescale 840 and information 850 relating to microreward cost is provided.

In some embodiments, body illustrations 810 can be provided from various viewpoints (including but not limited to front, back or side) or alternatively can be 3D rotating or rotatable illustrations. In some embodiments, a body illustration can be resized larger or smaller, or zoomed in focus on particular selected body areas (e.g head, face, torso, arms, hands, or legs, feet). In some embodiments, important bodily features can be colored or otherwise highlighted. In other embodiments, textual, voice, or graphical cues can be used to encourage touch input to body illustrations 810, as well as providing support for alternative textual or voice input by a patient.

In some embodiments, use case 820 can include but is not limited to, clinical trials, dose response, long term follow up, or pre-natal drug exposure. Long term follow up can include daily health status, pharmacovigilance procedures, follow up PGx registries, and health related quality of life indicators. Prenatal drug exposure related information can include information related to birth registries or cognitive outcomes.

Textual information relating data collection and automated data visualization text 830 can include prescription or placebo exposure, dose response, diagnosis (including timing, rarity, and type of diagnosis). Similarly, timing of mother, prenatal fetus, infant, or child exposure to pharmaceutical or health/cognitive outcomes can be tracked.

Time scale 840 can provide information regarding weeks of trial design and number of participants for both clinical trials or dose response. Long term follow up can track participation over year long time scales. Similarly, prenatal drug exposure monitoring can track participation over year time scales for both mother and child. In some embodiments, reward, microreward, or gamification features can be used to improve user engagement with the system. This can include but is not limited to game inspired application features that can engage user interest or provide favorable opportunities for socializing, learning, mastery, competition, achievement, improving status, or self-expression. Games can include games with random or semi-random output, skill based games, or both. Rewards for game can include awarding points, badges, placement on leaderboards or personal improvement graphs, or access to social or informational websites. Monetary rewards, including conventional monetary rewards, microrewards, coupons, or discounts can also form a part of the gamification experience. In other embodiments, the information 850 regarding microrewards or incentives used to encourage short or long term participation in monitored health trials can be provided by the custom touch diary mobile application 800. Such microrewards can include but are not limited to payment for a discrete test, and weekly, monthly, or yearly payments to encourage continued use of custom touch diary mobile application 800 to track symptoms. Payments can include cash or credit payments, rewards, coupons or discounts. In some embodiments, microrewards can include access to additional information or social media sites.

For purposes of illustration, programs and other executable program components are shown herein as discrete blocks, although it is understood that such programs and components may reside at various times in different storage components of local, server based, or cloud computing based systems and are executed by processor(s). Alternatively, the systems and procedures described herein can be implemented in hardware, or a combination of hardware, software, and/or firmware. For example, one or more application specific integrated circuits (ASICs) can be programmed to carry out one or more of the systems and procedures described herein.

Implementations of the systems, devices, and methods disclosed herein may comprise or utilize a special purpose or general-purpose computer including computer hardware, such as, for example, one or more processors and system memory, as discussed herein. Implementations within the scope of the present disclosure may also include physical and other computer-readable media for carrying or storing computer-executable instructions and/or data structures. Such computer-readable media can be any available media that can be accessed by a general purpose or special purpose computer system. Computer-readable media that store computer-executable instructions are computer storage media (devices). Computer-readable media that carry computer-executable instructions are transmission media. Thus, by way of example, and not limitation, implementations of the disclosure can comprise at least two distinctly different kinds of computer-readable media: computer storage media (devices) and transmission media.

Computer storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

An implementation of the devices, systems, and methods disclosed herein may communicate over a computer network. A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links, which can be used to carry desired program code means in the form of computer-executable instructions or data structures and which can be accessed by a general purpose or special purpose computer. Combinations of the above should also be included within the scope of computer-readable media.

Computer-executable instructions comprise, for example, instructions and data which, when executed at a processor, cause a general purpose computer, special purpose computer, or special purpose processing device to perform a certain function or group of functions. The computer executable instructions may be, for example, binaries, intermediate format instructions such as assembly language, or even source code. Although the subject matter is described in language specific to structural features and/or methodological acts, it is to be understood that the subject matter defined in the appended claims is not necessarily limited to the described features or acts described herein. Rather, the described features and acts are disclosed as example forms of implementing the claims.

Those skilled in the art will appreciate that the disclosure may be practiced in network computing environments with many types of computer system configurations, including personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, various storage devices, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Further, where appropriate, functions described herein can be performed in one or more of: hardware, software, firmware, digital components, or analog components. For example, one or more application specific integrated circuits (ASICs) can be programmed to carry out one or more of the systems and procedures described herein. Certain terms are used throughout the description and claims to refer to particular system components. As one skilled in the art will appreciate, components may be referred to by different names. This document does not intend to distinguish between components that differ in name, but not function.

It should be noted that the sensor embodiments discussed herein may comprise computer hardware, software, firmware, or any combination thereof to perform at least a portion of their functions. For example, a sensor may include computer code configured to be executed in one or more processors and may include hardware logic/electrical circuitry controlled by the computer code. These example devices are provided herein for purposes of illustration and are not intended to be limiting. Embodiments of the present disclosure may be implemented in further types of devices, as would be known to persons skilled in the relevant art(s).

At least some embodiments of the disclosure are directed to computer program products comprising such logic (e.g., in the form of software) stored on any computer useable medium. Such software, when executed in one or more data processing devices, causes a device to operate as described herein.

While various embodiments of the present disclosure are described herein, it should be understood that they are presented by way of example only, and not limitation. It will be apparent to persons skilled in the relevant art that various changes in form and detail can be made therein without departing from the spirit and scope of the disclosure. Thus, the breadth and scope of the present disclosure should not be limited by any of the described exemplary embodiments. The description herein is presented for the purposes of illustration and description. It is not intended to be exhaustive or to limit the disclosure to the precise form disclosed. Many modifications and variations are possible in light of the disclosed teaching. Further, it should be noted that any or all of the alternate implementations discussed herein may be used in any combination desired to form additional hybrid implementations of the disclosure.

1. A method for providing medical services, comprising
taking an image of a patient face;
determining position of facial features including eyes and eyebrows;
masking the eyes and verifying with a patient that the eyes are masked; and
identifying facial abnormalities, including at least one of rashes and alopecia.

2. The method of claim 1, wherein the image of the patient face is taken with a smartphone.

3. The method of claim 1, wherein masking the eyes in the image of the patient face is done with a smartphone.

4. The method of claim 1, wherein masking the eyes in the image of the patient face is done using a health management system.

5. The method of claim 1, wherein facial or hair abnormalities are symptoms of an auto-immune disease.

6. The method of claim 1, wherein facial or hair abnormalities are symptoms of lupus.

7. The method of claim 1, wherein machine learning is used at least in part to identify the facial or hair abnormalities.

8. The method of claim 1, wherein a convolutional neural network is used at least in part to identify the facial or hair abnormalities.

9. The method of claim 1, wherein identified facial or hair abnormalities are tracked.

10. The method of claim 1, wherein multiple patients are tracked.

11. A system for providing medical services, comprising
a diagnostic unit able to receive an image of a patient face, determine position of facial features including eyes and eyebrows, and identify facial or hair abnormalities, including at least one of rashes and alopecia;
an interactive unit that allows touch input to provide diagnostic information from the diagnostic unit.

12. The system of claim 11, wherein the interactive unit is a smartphone.

13. The system of claim 11, wherein the image of the patient face is taken with an interactive unit.

14. The system of claim 11, further comprising verifying masking the eyes in the image of the patient face using the diagnostic unit.

15. The system of claim 11, wherein facial or hair abnormalities are symptoms of an auto-immune disease.

16. The system of claim 11, wherein facial or hair abnormalities are symptoms of lupus.

17. The system of claim 11, wherein machine learning is used at least in part by the diagnostic unit to identify the facial or hair abnormalities.

18. The system of claim 11, wherein a convolutional neural network is used at least in part by the diagnostic unit to identify the facial or hair abnormalities.

19. The system of claim 11, wherein the identified facial or hair abnormalities are tracked.

20. The system of claim 11, wherein multiple patients are tracked.

21. A system for providing a custom medical diary, comprising
an interactive unit that allows touch mediated input to provide diagnostic information to the diagnostic unit;
a diagnostic unit able to receive touch mediated input related to physical symptoms of a patient and support a tracking history of the physical symptoms; wherein
the interactive unit can present at least one of a patient or a medical service provider with graphical and textual representation of symptom progression over time.

22. The system of claim 21, wherein the interactive unit is a smartphone.

23. The system of claim 21, wherein the interactive unit can take an image of a patient face and determine position of facial features including eyes and eyebrows.

24. The system of claim 21, wherein the interactive unit can use an image of a patient face with masked patient eyes and require verification with a patient that eyes are masked.

25. The system of claim 21, wherein the diagnostic unit can receive an image of a patient face, determine position of facial features including eyes and eyebrows, and identify facial or hair abnormalities, including at least one of rashes and alopecia.

26. The system of claim 21, wherein the diagnostic unit can align tracked physical symptoms with multi-organ outcome measures.
